(19) United States
(12) Patent Application Publication
(10) Pub. No.: US 2009/0054683 A1
(43) Pub. Date: Feb. 26, 2009

(54) Reactor and method for synthesising vinyl acetate in the gaseous phase

(76) Inventors: Karsten Bueker, Dortmund (DE); Steffen Schirrmeister, Muelheim an der Ruhr (DE); Bernd Langanke, Holzwickede (DE); Georg Markowz, Alzenau (DE); Ralf Hausmann, Wachtersbach (DE); Andreas Geisselmann, Offenbach (DE)

Correspondence Address: MARSHALL & MELHORN, LLC, FOUR SEAGATE - EIGHTH FLOOR, TOLEDO, OH, 43604, US

(21) Appl. No.: 11/665,079

(22) PCT Filed: Oct. 10, 2005
(86) PCT No.: PCT/EP05/10883

§ 371 Date: Oct. 30, 2008

(30) Foreign Application Priority Data
Oct. 15, 2004 (DE) 10 2004 050 585.3

(51) Int. Cl. C07C 67/00; B01J 19/00; B01J 23/44; B01J 23/58; B01J 23/10; B01J 23/34; B01J 21/06

(52) U.S. Cl. 560/231; 422/188; 422/196; 502/339; 502/330; 502/302; 502/304; 502/324; 502/326; 502/262

(57) ABSTRACT

The invention relates to a synthesis reactor and to a method for producing vinyl acetate, in which gaseous ethylene and acetic acid, in addition to oxygen or gases containing oxygen, react catalytically. The inventive synthesis reactor is a wall reactor, in which the catalytic synthesis takes place in a plurality of reaction chambers, whose free flow cross sections measure less than 2000 μm, preferably 1000 μm and whose indirectly cooled walls are coated with a palladium-gold catalyst.

The invention relates to a synthesis reactor for the production of monomeric vinyl acetate (VAM) in which gaseous ethylene and gaseous acetic acid as well as oxygen or gases containing oxygen react catalytically, the synthesis reactor being a wall reactor in which the catalytic synthesis takes place in a number of reaction chambers that are smaller than 2000 μm, preferably smaller than 1000 μm, in at least one dimension in relation to the free flow cross-section, the indirectly cooled walls of which are coated with a palladium-gold catalyst.

The prior art comprehensively describes the synthesis of vinyl acetate from ethylene. For this, ethylene, acetic acid and molecular oxygen or gases containing O₂, possibly with the addition of inert gases such as CO₂, for example, are brought to reaction in the presence of a catalyst at temperatures of 100° C. to 250° C. and increased pressure. This is done by passing the process gas over a catalyst bed. For this strongly exothermic reaction a catalyst containing palladium, gold and alkali metals on an oxidic carrier is normally used. The catalyst is in the form of moulded bodies, such as spheres, granulate, tablets or extrudates onto which the catalytically active substances are applied in a shell-shaped outer zone.

EP 1 106 247 B1 describes such a method and a suitable catalyst whereby the carrier catalyst has an ideal Pd proportion of 0.3 to 4.0% by weight and an Au content of 0.1 to 2.0% by weight. The thickness of the catalyst on the carrier is given as max. 1 mm, and is preferably less than 0.5 mm. EP 1 106 247 B1 cites productivities (designated as activity in the patent specification) of max. 225.5 g_{VAM}/kg_{cat}*h. Although high selectivity is mentioned in the patent specification, it is not described or quantified as a function of the selected carrier catalyst or the preparation method. In EP 0 987 058 B1 a silicon dioxide-based carrier catalyst is described which has a special carrier geometry.

Various synthesis catalysts which are known in the prior art are set forth in DE 190 20 390. In DE 198 34 569 A1 doping with hafnium is proposed which has resulted in high productivities of up to 1100 g_{VAM}/l_{cat}*h at 9 bars, 170° C. and 0.5% by weight Hf loading.

DE 199 14 066 discloses a catalyst in which barium or cadmium are used as doping elements and various metal oxides are used in the carrier body. The use of cadmium is detrimental on environmental grounds.

From DE 196 19 961, EP 0 916 402 A1 or EP 0 997 192 B1 optimising the external shape of the carrier bodies is also known. Mouldings, cylinders, ring and other shapes used in VAM synthesis are known. In EP 1 323 469 A3 a moulded body is described which as a pyrogenically produced silicon dioxide body has special open structures. In EP 1 106 247 B1 external dimensions of 2-15 mm are given for such moulded bodies. The loading is given as 0.2-1.5% by weight Au, 0.3-4.0% by weight Pd and 3.5-10% by weight potassium acetate.

In EP 0 997 291 B1 carrier materials are cited which comprise at least two components from the SiO₂, Al₂O₃, TiO₂ and ZrO₂ group. According to the aforementioned patent specification these carrier materials can be of any shape, e.g. cylinders, spheres or rings which are produced as extruded parts, extrudates or tablets. Also in EP 0 723 810 B1 a carrier material is disclosed that contains the elements zirconium and titanium. As an advantage of the invention the examples show that a productivity of 225 g_{VAM}/kg_{cat}*h is achieved.

It is also known and described in the prior art that a number of by-products are formed during the synthesis of VAM. In DE 199 20 390 the substances CO₂, ethyl acetate and high boilers such as ethylidene diacetate, ethylene glycol and its acetates or diacetoxyethylene are cited. In this patent specification a catalyst is described which beneficially influences selectivity with regard to the high boilers by adding vanadium.

It is also known that in the production of vinyl acetate considerable effort is required to separate the product from the educts and by-products following the synthesis stage. Separation normally takes place by means of post-synthesis distillation and other steps to separate the material streams. DE 39 34 614 A1 describes vinyl acetate synthesis with different processing stages for subsequent product treatment, wherein 1000-2000 ppm by weight is given as the known limit value for ethyl acetate and a target residual content of ethyl acetate of 150 ppm by weight is cited.

EP 0 072 484 discloses a method which through the addition of small quantities of H₂O into a vinyl acetate return flow and its introduction in the distillation plant leads to better dehydration. In DE 198 25 254 an analogous purification method is described in which in addition to water, acetic acid is added to the return stream, whereby a reduction in the ethyl acetate content is achieved. These methods are process stages that are downstream of the synthesis and reduce the formation of by-products through additional measures.

There is still a need for a method to enable high productivity as well as high selectivity of the catalyst. With regard to selectivity, there is a particular need for methods that suppress the formation of by-products, such as ethyl acetate, that are costly to separate.

The objective of the invention is therefore to overcome the deficiencies in the prior art and reduce the formation of by-products and at the same time achieve high selectivity and productivity. Here, productivity is defined as the mass of vinyl acetate formed per mass of catalyst and unit of time expressed in units of g_{VAM}/kg_{cat}*h, whereby the calculated mass of the catalyst does not contain a binding agent. The device and method according to the invention solve this problem in the prior art in that a synthesis reactor is used to produce vinyl acetate in which gaseous ethylene and acetic acid as well as oxygen or gases containing oxygen undergo a catalytic reaction, whereby a wall reactor is used as the synthesis reactor. This wall reactor has a number of reaction chambers in which the catalytic reaction takes place. At least one wall in each of these reaction chambers is coated with a catalyst and is indirectly cooled. The dimensions of the reaction chambers are selected in such a way that the free flow cross-section in each of these reaction chambers is less than 2000 μm, preferably less than 1000 μm in at least one dimension.

In an advantageous embodiment of the device the reaction chambers comprise a number of tubes or stacked plates which have a number of gaps whereby the tubes or gaps can be aligned in any way with regard to each other and ideally run parallel to each other.

It is an advantage if in an optimised embodiment only precisely one dimension of the tube-shaped reaction chambers is less than 2000 μm and preferably less than 1000 μm. In tube-shaped reaction chambers this is the free flow diameter and in gap-type reaction chambers either the height or the width of the gap of the free flow cross-section. The other dimensions, not being of these very small dimensions, have the advantage that seal-tightness, mechanical stresses and manufacturing processes can be more easily controlled.

The device according to the invention has a catalyst which contains palladium, gold and alkali metal compounds on an oxidic carrier material and is adhesively applied to the wall surfaces of the reaction chambers by means of a binding agent.

The palladium content of the catalyst according to the invention is 0.3 to 10% by weight and preferably 0.8 to 5% by weight, whereas the gold content is 0.20 to 5% by weight and preferably 0.4 to 2.5% by weight. Herein lies an essential advantage of the invention, namely that very much higher Pd and Au contents and thereby productivities can be achieved than in the case of the moulded body catalysts known from the prior art. On the one hand the reason for this is that due to the catalyst adhering to the reactor wall and the indirect wall cooling through the metal carrier, very advantageous heat dissipation is created.

On the other hand the metals can be distributed over the entire catalyst layer, since due to the small layer thickness and the high porosity of the carrier material there are no mass transport resistances during the reaction which have a negative effect on the activity. There is no shell structure as is usual in moulded bodies. The local noble metal concentration in the catalyst layer is not excessive as a result of the largely homogenous distribution so that the catalytically active metal surface as such is optimal and, accordingly, high activities are achieved.

Furthermore in the synthesis reactor according to the invention, a catalyst is used which has an alkali metal content of 0.4 to 6%, preferably 1 to 4% by weight. Potassium is the primary alkali metal used, which is preferred to exist in the form of its acetate.

In a further advantageous embodiment of the device according to the invention the catalyst used contains one or more elements from the group of earth alkali metals, lanthanoids, vanadium, iron, manganese, cobalt, nickel, copper, cerium, platinum, whereby the total proportion of these elements does not exceed 3% by weight.

The invention also covers the use of a catalyst in the synthesis reactor which contains an oxidic carrier material which as its principal component has an oxide from the group SiO₂, Al₂O₃, TiO₂ and ZrO₂. In an advantageous embodiment the carrier material contains further oxides as secondary components. Bentonites, for example, can be used as natural mixed oxides.

An advantageous embodiment variant of the device according to the invention involves the base material of the reaction chambers at least partially consisting of stainless steel and the catalyst being applied to the stainless steel walls to be coated with an oxidic or organic binding agent. For this, binding agents from the group of metal oxide sols, cellulose derivatives or alkali metal silicates, such as, for example, silicon oxide sols, methyl celluloses or water glass can be used.

The invention also covers a method using the aforementioned reactor in which the reactor is operated almost isothermally and the maximum temperature increase between the inlet and outlet of the synthesis reactor is 5 K, preferably 2 K. This small temperature increase not only applies to the difference between the inlet and outlet temperatures but also equally to all areas within the reactor. In the tubular reactors with catalyst beds used in the prior art, strong radial thermal gradients form in the individual reaction tubes, which cannot occur in the reactor according to the invention as the catalyst is present as a wall coating and complete optimal heat dissipation is ensured by the intensive indirect wall cooling.

The method according to the invention is also characterised in that in the reaction chambers the temperature is 100 to 250° C., preferably 150 to 200° C., with the pressure being in the range of 1 to 12 bars, preferably 6 to 10 bars absolute.

Advantageously the method according to the invention can be used in explosive process conditions with no restrictions with regard to the optimum operating parameters having to be taken into account because of explosive states being attained. Explosive conditions are taken to mean gas mixtures with a composition which could explode in a defined volume in the conditions present during the process, such as temperature and pressure. Of particular technical relevance is the lower explosion limit for oxygen which in U.S. Pat. No. 3,855,280 is typically quantified at 8%. With the method according to the invention process conditions are covered in which an O₂ content of over 7% by volume is present in the process gas.

The invention also covers a catalyst for use in a reactor for vinyl acetate synthesis characterised in that the palladium content of the catalyst is 0.5 to 10% by weight, preferably 0.8 to 5% by weight. In another advantageous composition of the catalyst the gold content of the catalyst is 0.25 to 5% by weight, preferably 0.4 to 2.5% by weight. In another advantageous composition of the catalyst according to the invention, the potassium content of the catalyst is 0.5 to 10% by weight and preferably 1 to 4% by weight.

In an advantageous embodiment the catalyst according to the invention also contains one or more elements from the group of earth alkali metals, lanthanoids, vanadium, iron, manganese, cobalt, nickel, copper, cerium, platinum whereby the total proportion of these elements does not exceed 3% by weight.

In an advantageous embodiment, the carrier material of the catalyst according to the invention contains an oxidic material with an oxide from the group SiO₂, Al₂O₃, TiO₂ and ZrO₂, whereby in an advantageous embodiment the carrier material contains further oxides as secondary components. Bentonites, for example, can be used as natural mixed oxides.

An advantageous further development consists in the catalyst layer being applied to the walls of the reaction chambers by means of an oxidic or organic binding agent, whereby the proportion of binding agent in the catalyst layer is 0-50% by weight and preferably binding agents from the group of metal oxide sols, cellulose derivatives or alkali metal silicates are used, for example, silicon oxide sols, methyl celluloses or water glass.

An optimised embodiment of the catalyst according to the invention consists in all the aforementioned doping and activation elements being homogeneously distributed over the entire volume of the catalyst layer without any layering as is the case in the shell catalysts known from the prior art.

To test the device according to the invention the catalyst was prepared in an analogue manner to the preparation in EP 1 008 385 A1 with a powder carrier containing SiO₂ being used identical in composition to the usual bulk catalyst carrier. The catalyst was then applied to two stainless steel plates with webs and activated with potassium acetate. The pairs of plates were connected to each other so that the catalyst layers were opposite each other with a gap of approx. 500 μm being formed. This pair of plates was placed in a pressure-resistant reactor heated by oil and operated almost completely isothermally at 155° C. The temperature increase was less than 1 K whereby measurements were taken at 5 measuring points along the path of flow.

The pressure in the reactor was in the range of 5 to 9 bars absolute. Ethylene, oxygen, methane and helium were added in gaseous form whereby the methane served as the internal standard for analysis. The acetic acid was added in liquid form and evaporated upstream of the reactor. The gas mixture entering the reactor was composed of the following, expressed in percent by volume: 63.2% ethylene, 5.7% oxygen, 4.2% methane, 9.6% helium and 17.2% acetic acid. Testing was initially started without oxygen, but over the course of one hour the oxygen content can rapidly be increased to the standard content. Analysis was carried out using gas chromatography and a CO₂ detector.

In an initial test, the results of which are set forth in Table 1, the dependence of the productivity on the palladium and gold content of the catalyst was investigated. The measurements were carried out at 5 bar and at 9 bar as well as with two different gas loads in the catalyst bed. The gas load was measured as product gas volume per mass of catalyst and time using the units l_{VAM}/kg_{cat}*h, whereby only the active catalyst mass was taken into consideration, i.e. without the binding agent proportion. A catalyst prepared according to the above method was used.

TABLE 1
Influence of the noble metal content on productivity

| | Productivity | Productivity |
|---|---|---|
| | [g_{VAM}/kg_{cat} * h] | [g_{VAM}/kg_{cat} * h] |
| Pd/Au content | at p = 5 bars and | at p = 9 bar and |
| in mass % | gas load = 12,000 l/kg * h | gas load = 24,000 l/kg * h |
| 0.83/0.36 | 450 | 750 |
| 2.50/1.10 | 830 | 1300 |
| 5.00/2.20 | 1120 | 2100 |

From Table 1 it can be seen that the productivity increases with increasing Pd/Au content. Although high values are sometimes reported in the prior art as being advantageous in principle, it can be assumed from the lack of appropriate tests or examples that temperatures cannot be controlled in the conventional method with such an active catalyst. An advantage of the invention can be seen here, namely that the temperature development as a result of a high Pd/Au loading no longer acts as a limit for the method.

In Test 2 the behaviour of the reactor according to the invention was investigated at various gas loads in the catalyst bed.

TABLE 2
Influence of the gas load of a Pd/Au wall catalyst
(0.83% Pd/0.36% Au) on productivity

| Gas load in l/kg * h | Productivity in |
|---|---|
| at 5 bar | g_{VAM}/kg_{cat} * h |
| 3000 | 300 |
| 6000 | 350 |
| 12000 | 400-450¹⁾ |
| 24000 | 450-480¹⁾ |

¹⁾For measurements see also Table 3

From Table 2 it can be seen that an advantage of the invention consists in operating the method at a gas load up to approx. 12,000 l/kg*h. Due to the deviation and disruption-free flow, the pressure loss in the reactor according to the invention is considerably less than in the known tubular reactor which has a catalyst bed. A further increase in the gas load above 12,000 l/kg*h does not bring about a significant increase in productivity.

Along with the findings from Test 3, in which increasing Pd/Au contents, increasing pressures and increasing gas loads were realised, it can be seen that all these three parameters have a positive effect on productivity, and their variations are not limited by the reactor or the method according to the invention.

TABLE 3
Influence of the operating pressure and the gas load on productivity with
different Pd contents

| | Productivity | Productivity | Productivity |
|---|---|---|---|
| | [g_{VAM}/kg_{cat} * h] | [g_{VAM}/kg_{cat} * h] | [g_{VAM}/kg_{Kat} * h] |
| | pressure 5 bars | pressure 5 bars | pressure 9 bars |
| Pd/Au content | gas load = | gas load = | gas load = |
| in mass % | 12,000 l/kg * h | 24,000 l/kg * h | 24,000 l/kg * h |
| 0.83/0.36 | 450 | 480 | 760 |
| 2.50/1.10 | 830 | 850 | 1350 |
| 5.00/2.20 | 1120 | not measured | 2100 |

In Test 4 the temperature dependence of the method was investigated whereby the selectivity for vinyl acetate was considered in relation to ethylene and the productivity was considered in relation to vinyl acetate. It was found that with regard to selectivity an optimum is passed, which is in the range of 160° C. to 170° C. in the case of a pressure of 9 bars and a selected catalyst loading of 0.83% by weight Pd and 0.36% by weight Au, for example, whereby selectivities of almost 98% are achievable. Productivity is also increased with further increases in temperature so that a productivity of up to 1,400 g_{VAM}/kg_{cat}*h was achieved at 185° C. at this low Pd/Au loading.

TABLE 4
Influence of the operating temperature at p = 9 bars and gas load =
24,000 l/kg * h (wall catalyst with 0.83% Pd/0.36% Au)

| | T = | T = | T = | T = |
|---|---|---|---|---|
| | 155° C. | 165° C. | 175° C. | 185° C. |
| VAM Productivity | 700 | 900 | 1150 | 1400 |
| g_{VAM}/kg_{cat} * h | | | | |
| VAM Selectivity in | 97.4 | 97.4 | 96.8 | 95.6 |
| % (related to C₂H₄) | | | | |

In Test 5 an attempt was made to use the method according to the invention under explosive gas conditions by increasing the oxygen concentration. Test conditions 3 and 4 are within the explosive range with regard to O₂/C₂H₄ ratios. An advantage of the invention can be seen in the fact that in order to increase productivity, explosive process conditions can be intentionally set in order to optimise the method.

TABLE 5
Effect of increasing the O₂ concentration on the productivity of VAM
at p = 5 bars and gas load = 12,000 l/kg * h (wall catalyst with 0.83%
Pd/0.36% Au)

| | Test | | | |
|---|---|---|---|---|
| | condition | Test | Test | Test |
| | 1 | condition 2 | condition 3 | condition 4 |
| % by volume | 63.2 | 61.2 | 59.5 | 57.8 |
| C₂H₄ | | | | |
| % by volume | 6.0 | 8.7 | 11.3 | 13.8 |
| O₂ | | | | |
| % by volume | 17.2 | 16.7 | 16.2 | 15.7 |
| HOAc | | | | |
| Inerts (He, CH₄) | 13.9 | 13.5 | 13.1 | 12.7 |
| Space-time yield | 430 | 630 | 700 | 1100 |
| VAM | | | | |
| g_{VAM}/kg_{cat} * h | | | | |

In Test 6 the influence of the layer thickness on the productivity and area-time yield of vinyl acetate was investigated. The almost constant mass-specific activity of the catalyst at increased layer thickness shows that mass transport resistances play only a subordinate role within the layer. A further advantage of the invention is therefore that there is no need for shell-like noble metal distribution, which is associated with high local noble metal concentrations and thereby low noble metal surface areas.

TABLE 6
Influence of the layer thickness of the wall catalyst with 2.5% by
mass Pd on the area-time yield of VAM

| Layer | Productivity | Area-time |
|---|---|---|
| thickness | VAM | yield VAM |
| in μm | g_{VAM}/kg_{cat} * h | g_{VAM}/m²_{cat} |
| 300 | 850 | 130 |
| 500 | 780 | 196 |

With higher loading of the wall catalyst, a VAM productivity of up to 5 kg_{VAM}/kg_{cat}*h was observed at pressures of up to 9 bars.

In a further series of tests a Pd/Au catalyst was used which was prepared according to the method described in EP 0 723 810 A1. In contrast to the moulded bodies described in EP 0 723 810 A1 particles with a particle size of 50-150 μm were used for the tests described below. The basic material is identical to that of the moulded body. The ethylene concentration in the educt flow was reduced in relation to tests 1 to 6 and replaced with a corresponding volumetric proportion of inert gas and a small proportion of water vapour. Changing the proportion of ethylene in the educt stream to the extent carried out had a negligible influence on the synthesis reaction. In Test 7, the wall catalyst prepared according to the above method was used, said catalyst containing 2.5% by weight Pd and 1.1% by weight Au. As a further test condition a gas temperature of 155° C. and a pressure of 9 bars were set. The composition of the educt gas was selected as follows:

49.2% by vol. ethylene (C₂H₄)

18.0% by vol. acetic acid (HOAc)

1.3% by vol. water (H₂O)

31.5% by vol. oxygen and inert gas (helium+methane)

The results of measurement are set forth in Table 7.

TABLE 7
Influence of the O₂ concentration on productivity and selectivity

| | | | Selectivity VAM | C₂H₄ |
|---|---|---|---|---|
| O₂ (%) | Gas load | Productivity | (related to C₂H₄) | conversion |
| Vol % | l/kg_{cat} * h | g_{VAM}/(kg_{cat} * h) | % | % |
| 6.5 | 6000 | 1600 | 94.3 | 15.0 |
| 9.0 | 6000 | 1900 | 94.7 | 17.6 |
| 11.5 | 6000 | 2320 | 93.6 | 21.5 |
| 14.0 | 6000 | 2620 | 93.4 | 24.8 |
| 14.0 | 12000 | 3180 | 96.0 | 14.2 |

Test 7 shows that, with a space velocity of 6,000 l/kg_{cat}*h, by increasing the oxygen concentration from 6.5% by volume, which is not in the explosive range, to 14.0% by volume in the explosion range, the conversion of ethylene increases from 15% to 24.8%. In this case it is really surprising that this marked increase in conversion only leads to a very small decrease in selectivity from 94.3 to 93.6%. In actual fact with the very high O₂ concentration in the explosive range, much greater CO₂ formation due to the parallel reaction of ethylene with O₂ is expected here, but surprisingly this did not happen. Overall there is an increase in productivity from 1600 g_{VAM}/kg_{cat}*h to 2620 g_{VAM}/kg_{cat}*h.

With an increase in the space velocity from 6000 to 12,000 l/kg_{cat}*h, the productivity can be increased further to 3180 g_{VAM}/kg_{cat}*h and selectivity with regard to ethylene increases from 93.4 to 96% compared with operation at 6000 l/kg_{cat}*h.

In Test 8 the method known from the prior art using a bulk catalyst in a tubular reactor was compared with the method according to the invention using a microreactor. As in Test 7 the wall catalyst prepared according to the above method was used and also contained 1.1% by weight Au. The gas temperature was 155° C. and the pressure was 9 bars, with a gas load of 12,000 l/kg_{cat}*h being selected.

The composition of the educt gas was selected as follows:

TABLE 8
Comparison between wall catalyst and bulk catalyst with normal Pd
loading of the different catalysts in each case

| | | | |
|---|---|---|---|
| 49.2% by vol. | Ethylene (C₂H₄) | 6.5% by vol. | Oxygen |
| 18.0% by vol. | Acetic acid (HOAc) | 25.0% by vol. | Inert gas |
| 1.3% by vol. | Water (H₂O) | | (Helium + |
| | | | Methane) |

| | | Selectivity | |
|---|---|---|---|
| | | VAM (related | C₂H₄ |
| | Productivity | to C₂H₄) | conversion |
| | [g_{VAM}/kg_{cat} * h] | [%] | [%] |
| Wall catalyst with | 2050 | 96.3 | 9.6 |
| 2.5% by weight Pd | | | |
| Bulk catalyst with | 720 | 94.1 | 3.2 |
| 0.56% by weight Pd | | | |

With increased C₂H₄ conversion, the wall catalyst also surprisingly exhibits greater VAM selectivity with regard to ethylene than does the bulk catalyst. Overall, this results in a wall catalyst productivity of 2050 g_{VAM}/kg_{cat}*h compared with 720 g_{VAM}/kg_{cat}*h for the bulk catalyst. For the wall catalyst, a metal loading was able to be selected which is not achievable for the bulk catalyst since such a high Pd and Au loading brings about local overheating, known as “hot spots”.

In Test 8 the by-product spectrum of the method according to the invention and the known method was investigated.

TABLE 9
Comparison of the by-product spectra: T = 155° C., p = 9 bars,
gas load = 12,000 l/kgcat * h

| | Wall catalyst | Bulk catalyst |
|---|---|---|
| | mg_{by-product}/kg_{VAM} | mg_{by-product}/kg_{VAM} |
| By-product | [ppm] | [ppm] |
| Acetaldehyde | 5774 | 8917 |
| Methyl acetate | 137 | 1076 |
| Ethyl acetate | 1931 | 4283 |
| 1,2-Ethanediol onoacetate | 907 | 7819 |
| 1,1-Ethanediol diacetate/ | 868 | 1691 |
| Ethylidene diacetate | | |
| 1,1-Ethene dioldiacetate/ | 2038 | 7731 |
| Vinylidene diacetate | | |
| Total 1,2-diacetates | 2906 | 9422 |

Surprisingly, the comparison of the wall catalyst with the bulk catalyst shows that, in the case of the wall catalyst, by-product formation is considerably lower than in the case of the bulk catalyst. This constitutes an important economic advantage as the costs of cleaning and separating the product and by-products are considerably reduced in an industrial application using the device according to the invention.

In a further Test 9 the service life of the catalyst was measured using the fall in productivity. The test conditions with respect to the composition of the educt and the temperature were identical to Test 8; only the pressure for the test was 9.5 bars and different gas loads were selected differently for the wall catalyst and tubular reactor.

TABLE 10
Comparison of the service life of wall and bulk catalysts

| | | | |
|---|---|---|---|
| Reactor with wall catalyst | 2.5% Pd | gas load = | 12,000 l/kg_{cat} * h |
| Tubular reactor with bulk | 0.56% Pd | gas load = | 6,000 l/kg_{cat} * h |
| catalyst | | | |

| | Wall catalyst | | Bulk catalyst | |
|---|---|---|---|---|
| | Productivity | Relative | Productivity | Relative |
| Service | [g_{VAM}/ | productivity | [g_{VAM}/ | productivity |
| life [h] | kg_{cat} * h] | (10 h = 100%) | kg_{cat} * h] | (10 h = 100%) |
| 10 | 2100 | 100 | 390 | 100 |
| 20 | 2150 | 102 | 370 | 95 |
| 100 | 2000 | 95 | 270 | 69 |

The productivity value after 10 hours in each case was taken as the baseline value and the relative change with regard to this baseline value is set forth in Table 10. The measurements show the surprising result that over a duration of 100 hours the wall catalyst is hardly deactivated compared to the bulk catalyst in spite of clearly greater productivity. For technical applications, this represents a considerable advantage of the wall catalyst over the bulk catalyst.

Surprisingly it was found that a selectivity of up to approx. 98% could be achieved with regard to ethylene at the same time as very high conversion rates. In addition to this it was surprising to observe that a clearly reduced quantity of by-products was formed. The high vinyl acetate selectivity and the low rate of by-product formation consequently result in a considerably reduced need for purification of the product with the method according to the invention in comparison with the method according to the prior art and thus to significant economic advantages.

1-21. (canceled)

22. A synthesis reactor for producing vinyl acetate in which gaseous ethylene and acetic acid as well as oxygen or gases containing oxygen undergo a catalytic reaction, wherein the synthesis reactor is a wall reactor and the catalytic synthesis takes place in a number of reaction chambers, whereby the free flow cross-section in each of these reaction chambers is less than 2000 μm in at least one dimension and at least one wall of the reaction chambers is coated with catalyst and at least one wall of the reaction chambers is indirectly cooled.

23. The reactor according to claim 22, wherein precisely one dimension of the reaction chambers is less than 2000 μm.

24. The reactor according to claim 22, wherein the reaction chambers comprise a number of tubes or gaps arranged by way of plates, whereby these can be aligned in any direction.

25. The reactor according to claim 22, wherein the catalyst contains palladium, gold and alkali metal compounds and is adhesively applied to the wall surfaces of the reaction chamber by way of a binding agent.

26. The reactor according to claim 22, wherein the palladium content of the catalyst is 0.5 to 10 percent by weight.

27. The reactor according to claim 22, wherein the gold content of the catalyst is 0.20 to 5 percent by weight and preferably 0.4 to 2.5 percent by weight.

28. The reactor according to claim 22, wherein the potassium content of the catalyst is 0.5 to 10 percent by weight.

29. The reactor according to claim 22, wherein the catalyst used contains one or more elements selected from the group consisting of: earth alkali metals, lanthanoids, vanadium, iron, manganese, cobalt, nickel, copper, cerium, and platinum;
whereby the total proportion of these elements does not exceed 3% by weight.

30. The reactor according to claim 22, wherein the catalyst used contains an oxidic metal carrier, with a metal oxide selected from the group consisting of: SiO₂, Al₂O₃, TiO₂ and ZrO₂ as the principal component;
whereby in an advantageous embodiment the carrier material contains further oxides as secondary components and the carrier material can be a natural mixed oxide from the group of bentonites.

31. The reactor according to claim 22, wherein the catalyst used is applied to the walls of the reaction chambers using an oxidic or organic binding agent, whereby binding agents from the groups metal oxide sols, cellulose derivatives or alkali metal silicates, such as silicon oxide sols, methyl celluloses or water glass are used.

32. The reactor according to claim 22, wherein the basic material of the reaction chambers comprises, at least partially, a stainless steel.

33. A method of using the reactor according to claim 22, wherein the reactor is operated isothermally with a maximum temperature increase between the inlet and outlet of the synthesis reactor of 5 K.

34. The method according to claim 33, wherein the temperature in the reaction chambers is 100 to 250° C., with the pressure being in the range of 0 to 12 bars.

35. The method according to claim 33, wherein it is carried out in explosive process conditions wherein the oxygen content in the process gas is above 7% by volume.

36. A catalyst for use in the synthesis reactor according to claim 25, wherein the palladium content of the catalyst is 0.5 to 10% by weight.

37. The catalyst according to claim 36, wherein the gold content of the catalyst is 0.25 to 5% by weight.

38. The catalyst according to claim 36, wherein the potassium content of the catalyst is 0.5 to 10 percent by weight.

39. The catalyst according to claim 36, comprising at least one material selected from the group consisting of: earth alkali metals, lanthanoids, vanadium, iron, manganese, cobalt, nickel, copper, cerium, and platinum; whereby the total proportion of these elements does not exceed 3% by weight.

40. The catalyst according to claim 36, comprising an oxidic carrier material with a metal oxide selected from the group consisting of: SiO₂, Al₂O₃, TiO₂ and ZrO₂; whereby in an advantageous embodiment the carrier material contains further oxides as secondary components. Bentonites, for example, can be used as natural mixed oxides.

41. The catalyst according to claim 36, wherein it can be applied to the walls of the reaction chambers by way of an oxidic or organic binding agent, whereby binding agents from the groups metal oxide sols, cellulose derivatives or alkali metal silicates, such as, for example, silicon oxide sols, methyl celluloses or water glass, are preferred to be used.

42. The catalyst according to claim 36, wherein all doping and activation elements are homogeneously distributed in the entire volume of the catalyst layer.

43. The reactor according to claim 23, wherein the measured dimension of the reaction chambers is less than 1000 μm.

44. The reactor according to claim 24, wherein the tubes or gaps are parallel to each other.

45. The reactor according to claim 26, wherein the palladium content of the catalyst is 0.8 to 5 percent by weight.

46. The reactor according to claim 27, wherein the gold content of the catalyst is 0.20 to 5 percent by weight.

47. The reactor according to claim 28, wherein the potassium content of the catalyst is 1 to 4 percent by weight.

48. A method of using the reactor according to claim 33, wherein the maximum temperature increase between the inlet and outlet of the synthesis reactor is 2 K.

49. The Method according to claim 33, wherein the temperature in the reaction chambers is 150 to 200° C., with the pressure being in the range of 6 to 10 bars.

50. A catalyst for use in a synthesis reactor according to claim 22, wherein the palladium content of the catalyst is 0.8-5% by weight.

51. The catalyst according to claim 37, wherein the gold content of the catalyst is 0.4 to 2.5% by weight.

52. The catalyst according to claim 36, wherein the gold content of the catalyst is 0.4 to 2.5% by weight.

53. The catalyst according to claim 36, wherein the potassium content of the catalyst is 0.5 to 10 percent by weight and preferably 1 to 4 percent by weight.

54. The catalyst according to claim 36, wherein the carrier material contains further oxides as secondary components. Bentonites, for example, can be used as natural mixed oxides.

55. The catalyst according to claim 36, wherein Bentonites are used as natural mixed oxides.

56. The catalyst according to claim 36, wherein the binding agents are selected from the group consisting of: metal oxide sols, cellulose derivatives or alkali metal silicates.
